(19) United States
(12) Patent Application Publication
(10) Pub. No.: US 2005/0245620 A1
(43) Pub. Date: Nov. 3, 2005

(54) Fast startup in autothermal reformers

(76) Inventors: Yanlong Shi, Littleton, MA (US); Jian Lian Zhao, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/534,685

(22) PCT Filed: Nov. 13, 2003
(86) PCT No.: PCT/US03/36258

§ 371 Date: Jul. 1, 2005

(51) Int. Cl. C07C 27/06

(52) U.S. Cl. 518/716

(57) ABSTRACT

Startup of autothermal reformers for hydrogen production in simplified by providing a first, ATR catalyst bed (14) containing a low-temperature-lightoff catalyst and a second catalyst in a second bed (16), or in a second part of the first bed (14), which is a better steam reforming catalyst. The combination is an effective solution for the startup needs of small or mobile steam reformers.

### RELATED APPLICATION(S)

This application claims the benefit of U.S. Provisional Application No. 60/425,954, filed Nov. 13, 2002, the entire teachings of which are incorporated herein by reference.

### GOVERNMENT SUPPORT

The invention was supported, in whole or in part, by a grant FC02-99EE50580 from the U.S. Department of Energy. The Government has certain rights in the invention.

### BACKGROUND OF THE INVENTION

Autothermal reformers (ATRS) are one of the principal types of reactor for generating hydrogen via the steam reforming of fuel and water into a mixture containing hydrogen and other gases. The steam reforming reaction is well known. In this reaction, a fuel in gaseous form, typically a hydrocarbon or an alcohol, is mixed with steam at elevated temperature, usually in the presence of a catalyst. The fuel and water are converted into hydrogen and carbon monoxide. Using methane as an example, the “reforming” reaction is:
CH₄+H₂O→CO+3H₂
In subsequent reactions the CO (carbon monoxide) is usually reacted with more water to form CO₂ (carbon dioxide) and H₂. This is called the “water gas shift” reaction:
CO+H₂O⇄CO₂+H₂
The resulting hydrogen-containing gas, generally called the reformate, is used for any of several purposes, but particularly for the generation of electricity using a fuel cell.

The steam reforming reaction is endothermic (absorbs heat), and so heat must be supplied to the system to drive the reaction. In “pure” steam reforming, the heat is supplied from an outside source to the catalyst bed. This is typically done by combusting a fuel, although other heat sources can be used. In the combustion of methane, for example,
CH₄+2O₂→CO₂+2H₂O+heat.
The heat needed for reforming amounts to about the combustion of one methane for every three reformed. (Similar ratios apply to other fuels.)

The need to transport heat from an outside source to a catalyst bed can be an obstacle to rapid startup or rapid change of operating parameters in a steam reformer. This can be avoided by the use of other forms of steam reforming, most commonly called “autothermal reforming”, or “partial oxidation reforming”. In autothermal reforming, fuel, steam and also a controlled amount of air are mixed and injected into the reactor. The oxygen in the air then reacts with some of the fuel, usually in the presence of a catalyst, thereby producing heat. The heat is absorbed by the reforming reaction of fuel and steam, as described above, which is occurring at the same time in the same catalyst bed. Once the system is at operating temperature, the amount of heat required for the reforming reaction can be generated in a controlled way by controlling the ratio of the inlet air to the amount of fuel being reformed.

However, the system must initially be heated, so that the catalyst reaches an effective operating temperature. It therefore has required preheating by hot gas, or electricity, or by pre-combustion or local ignition, to start up a cold ATR reactor. While this is not difficult in a large, fixed chemical plant, it is much more difficult in a mobile reformer, for example in a vehicle, or in a small reformer at a non-industrial site, such as in a distributed electric power generating system. Such small reformers often need to undergo cold startup several or many times per day. In these reformers, the ATR type of reaction is useful because creating the required heat in the bed itself is much quicker than supplying it via heat transfer through a heat exchanger. The problem still remains of how to start the reaction in the first place. Clearly heat must be supplied from some other source, such as combustion external to the reforming bed, or via electrical heating of the catalyst, to raise the temperature enough to start the autothermal reforming reaction. It is therefore useful to have a catalyst that will begin effective catalytic activity at as low a temperature as possible (have a low “light-off” temperature). In particular, a low light-off temperature reduces the need for external combustion or electricity. Once the catalyst reaches light-off temperature, the heat created by the oxidation portion of the ATR reaction is sufficient to maintain or increase the catalyst's activity, as well as to heat downstream catalyst and to provide enough heat for the reforming reaction.

The ATR catalyst must fulfill several requirements, which at present conflict to some extent. In addition to having as low a low light-off temperature as feasible, the catalyst must efficiently convert all components of the fuel into hydrogen after lighting off. A few fuels are nearly mono-component (e.g., natural gas; some alcohols), but many practically important fuels, particularly gasoline and other petroleum derivatives, are mixtures of many components. Gasoline, for example, contains both light alkanes, such as octanes, and aromatic compounds, such as napthalenes. The latter are considerably more difficult to reform, and are very demanding in terms of catalyst properties. These properties are shared by most petroleum products (kerosine, naptha, jet fuel etc.) and by many natural or refined feedstocks, including liquids from coal and tar, and the like.

At present, the best catalysts for aromatics and other difficult fuels have light-off temperatures in the range of 350 deg. C. or more. On the other hand, catalysts are known that will light off as low as about 150 deg. C., but do not reform heavy components well under normal operating conditions, displaying fuel “slip” (bypass) and/or poor selectivity for hydrogen formation. No single catalyst is known at present that is satisfactory in both of these aspects.

### SUMMARY OF THE INVENTION

A catalyst for rapid startup of an autothermal reformer (ATR) comprises at least two catalyst portions in sequence in an ATR reactor. A first, upstream catalyst portion is selected to have a comparatively low light-off temperature. A second catalyst portion, downstream of the first, is selected for optimal reforming of the fuel, and in particular of aromatics or other species in the fuel that are difficult to reform completely. The catalysts may be in sequential containers. More preferably, they are layered in a common bed for optimization of heat transfer from the first catalyst to the second catalyst. More preferably, the catalysts are monoliths, and the monoliths are mounted in a common housing for direct transfer of heat from the first, low-light-off catalyst to the second catalyst.

The catalysts types may also be mixed, particularly in a granular bed. However, it is preferred to have the upstream portion of the bed enriched in the first catalyst, and to have the second catalyst predominate in the most downstream portion of the bed, in order to take maximum advantage of the distinctive properties of the catalysts. Hence, granular or pelleted catalysts may be packed into the reactor in two layers, but some mixing the interface is not detrimental. A more complex gradient is typically used only if experimental results show that it is more effective. (Simple layering will tend to be effective because heat generated by the low-lightoff catalyst flows downstream to the higher lightoff catalyst.) With catalysts on monolithic supports, the supports are positioned in the reactor in the appropriate order. Gradient monoliths are difficult to construct with standard techniques, but multiple layers of monolith with successively varying ratios of the two catalysts could also be used.

In one aspect of the invention, a catalyst system for rapid startup of an autothermal reformer comprises a first, upstream portion having at least a majority of a first catalyst having a first lightoff temperature, and a second, downstream portion having at least a majority of a second catalyst having a second, higher lightoff temperature, wherein the difference between the first and second lightoff temperatures is at least about 25 deg. C. The respective catalysts can be housed in a common housing, or they can be housed in separate housings, with a gas flow path connecting the first housing to the second housing.

In another aspect, a method for providing rapid startup in an autothermal reforming reaction comprises providing a first catalyst portion for conducting an autothermal reforming reaction, the first catalyst portion having a first lightoff temperature; providing a second catalyst portion for conducting an autothermal reforming reaction in fluid communication with the first catalyst portion, the second catalyst portion having a second lightoff temperature that is at least 25 deg. C. higher than the first lightoff temperature; heating at least part of the first catalyst portion to the first lightoff temperature; and flowing a mixture comprising at least air and fuel over the heated first catalyst portion to create heat by reaction of the air and fuel.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows catalyst bed temperatures and hydrogen output plotted as a function of time for a low lightoff temperature catalyst pellet bed under simulated ATR conditions;

FIG. 2 shows catalyst bed temperatures plotted as a function of time for a low lightoff temperature catalyst coated onto a metal monolith under simulated ATR conditions;

FIG. 3 shows catalyst bed temperatures as a function of time for a low lightoff temperature catalyst and a high lightoff temperature catalyst under simulated ATR conditions; and

FIGS. 4A and 4B are schematic illustrations of autothermal reformers employing the catalyst system of the present invention.

### DETAILED DESCRIPTION OF THE INVENTION

A description of preferred embodiments of the invention follows.

Demonstration of the effectiveness of the combination of the invention may be confirmed by simple experimentation, similar in scope to the experimentation required to select the appropriate amount and geometric arrangement of any catalyst for a given process flow. A sample of a low light-off temperature catalyst is obtained from a supplier, or synthesized. Its light-off temperature is measured. Its ability to reform more difficult hydrocarbons in a mixture, for example gasoline, is also evaluated, typically at various temperatures. Standard evaluation methods are used. These include use of analytical instrumentation to determine completeness of conversion of the fuel; use of temperature sensors at various places in the apparatus; and variation of flow rate to then determine the functional capacity of the catalyst. The results may be used to model the behavior of the catalyst, so that the effects of variations in bed volume and geometry, etc., can be calculated.

Likewise, a sample of a high light-off temperature high efficiency reforming catalyst is obtained or made. Its efficiency at completely reforming difficult fuels, such as gasoline and the like, is measured using similar procedures, particularly as a function of temperature.

The two catalysts are placed in order in an ATR catalyst bed, with the first low light-off catalyst arranged upstream of the second high light-off catalyst. The relative thicknesses of the two catalysts can be estimated from models, and confirmed or adjusted by experiment. The presence of the low lightoff catalyst will cause the catalyst bed to warm up to the desired operating temperature significantly faster than can be obtained with the high lightoff catalyst alone. This is found despite the likelihood that the desired operating temperature, usually 600 deg. C. or more, is typically higher than the lightoff temperature of either catalyst. As the bed is initially heated by another source, such as hot gas from a burner, the low lightoff catalyst will begin to operate much sooner than will the high lightoff catalyst, and will rapidly raise bed temperature to a level at which both catalysts are operative and generating heat. The major time savings is in the difference in the time required under external heating to get the low-light off catalyst up to ignition temperature, as opposed to the high light-off catalyst.

Types of catalysts can be compared, and preferred catalyst pairs selected. Testing of additional batches of the selected catalysts will normally be done. The efficacy of the paired catalysts will normally be evident from the analytical data collected—i.e., faster temperature rise, with complete conversion of all species at temperature, with minimum volume. A more dramatic difference will be seen by reversing the order of the catalysts, putting the high lightoff catalyst first. The resulting bed will typically compare unfavorably with either catalyst alone, because it will light off no faster, or not greatly faster, than the high lightoff catalyst alone, while the conversion efficiency for difficult species of fuel will typically not be as good as that of an equivalent volume composed entirely of high lightoff high-efficiency catalyst.

Examples of Potentially Suitable Materials

High-efficiency, high-lightoff temperature catalysts are known, and include commercially available materials. Detailed compositions of catalysts are often proprietary to their manufacturers, so that selection is typically done by product name rather than detailed chemical composition. In the experiments shown below, a catalyst was obtained from the dnic² unit of the OMG AG & Co. (Postfach ¹³⁵¹, D-63403 Hanau, Rodenbacher Chaussee 4, D-63457 Hanau-Wolfgang, Germany) as type 383.

Low temperature lightoff materials are less common—partially because of the difficulty of achieving high efficiency with these materials. Compositions with low lightoff temperatures have been described in U.S. Pat. No. 6,110,861 to Krumpelt et al. A sample of this material in pellet form was obtained from ANL (Argonne National Laboratories). A sample of material believed to be substantially similar was prepared by Sud-Chemie, Inc. (P.O. Box 32370, Louisville, Ky.) on a FeCrAloy® metal monolith as sample FCR-HCl. These catalysts had a lightoff temperature in the range of about 150-180 deg. C. for fuel reforming, and had reasonable catalytic efficiency except for the aromatics and similar complex species in gasoline.

Comparison of the lightoff properties of the above catalysts are shown in the figures. These catalysts are potentially suitable for forming a catalyst pair that will perform according to the invention. In the experiments, small cylinders of catalyst—either pellets or monoliths—are placed in a holder and fed with simulated reformate, air and steam. The gasoline is vaporized and preheated to about 140 deg. C. The steam and fuel are premised and heated in another apparatus (typically, a coil wrapped with heating tape) to about 140 deg. C., in the experiments shown. The two gas streams are mixed and fed to the catalyst. The catalyst is in a “fimuace” made by surrounding it with an electrically-powered radiant heater. The power to the heater is selected to warm the catalyst bed until lightoff of the catalysts is obtained over a particular time. This can be a prolonged period, depending on the power selected; most of the warm-up period is not shown in the data below. In the figure legends, WGHSV is wet gas hourly space velocity. Phi (Φ) is the ratio of the air theoretically required to oxidize all the fuel to the amount of air actually supplied; in effect, the reciprocal of the percentage of fuel burned.

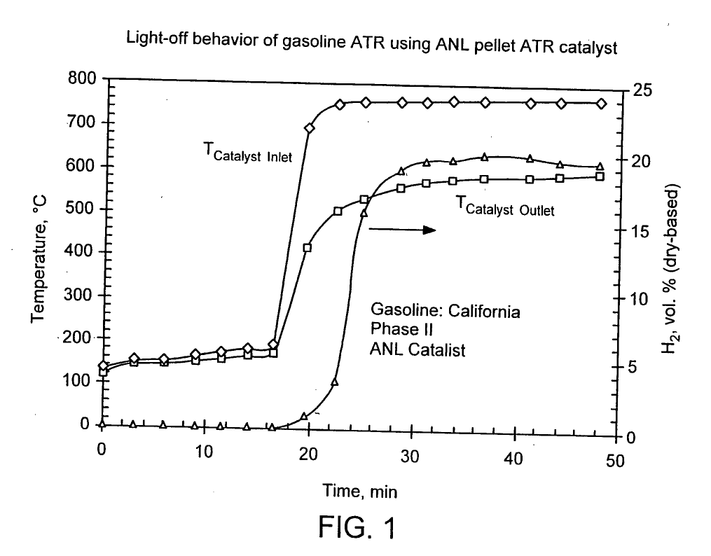

In FIG. 1, the properties of a sample of low lightoff temperature catalyst, specifically the low lightoff catalyst pellet from Argonne National Laboratories (ANL) mentioned above, are shown by way of a plot of catalyst bed inlet and outlet temperature, and amount of hydrogen production, as a function of time. In the experiment, a mixture of vaporized fuel, air and steam is fed into a reactor bed, and the catalyst is gradually raised in temperature to simulate the operation of a reformer. The temperatures of the catalyst bed inlet and outlet are shown (diamonds and squares, respectively), and the yield of hydrogen observed is shown (triangles). The temperatures of the inlet and outlet are the same, until a temperature in the bed of about 180 deg. C. is reached, at about 18 minutes of heating. Then oxidation begins, and the inlet temperature rapidly rises to about 750 deg. C., while the outlet temperature rises more gradually to about 620 deg. C. This rise is accompanied by the appearance of hydrogen at the outlet (triangles; right vertical axis). By about thirty minutes, the bed is close to equilibrium, as seen in the plateauing of the outlet temperature and the hydrogen concentration. The difference in the inlet and outlet temperatures reflects the heat that is drawn from the catalyst bed by the reforming reaction.

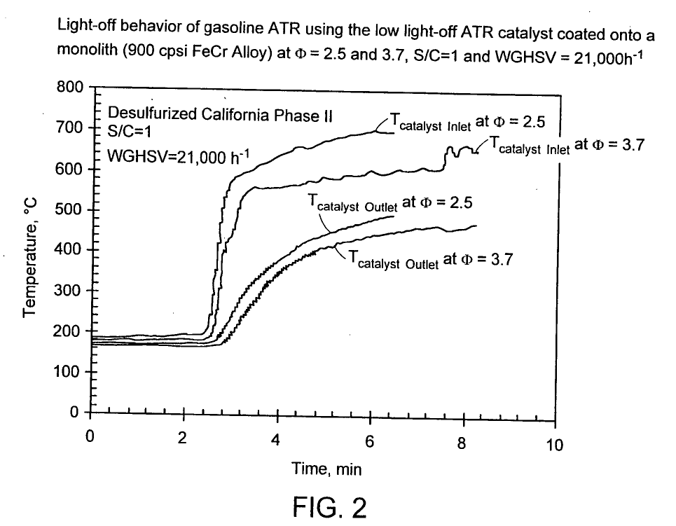

FIG. 1 was conducted on a pellet bed. In FIG. 2, the same ANL catalyst is coated onto a metal monolith, which has less thermal mass and better thermal conductivity. The four curves shown are, from the highest down, the inlet temperature at phi=2.5; inlet at phi=3.7; outlet at phi=2.5; and outlet at phi=3.7. The value of 3.7 is theoretically correct; the value of 2.5 reflects having additional air present for faster warmup, resulting in additional oxidation of fuel during the warmup stage.

With 200 deg. C. heating applied to the incoming reactants, FIG. 2 shows that lightoff is achieved rapidly, and warmup is nearly complete at about 6 to 8 minutes.

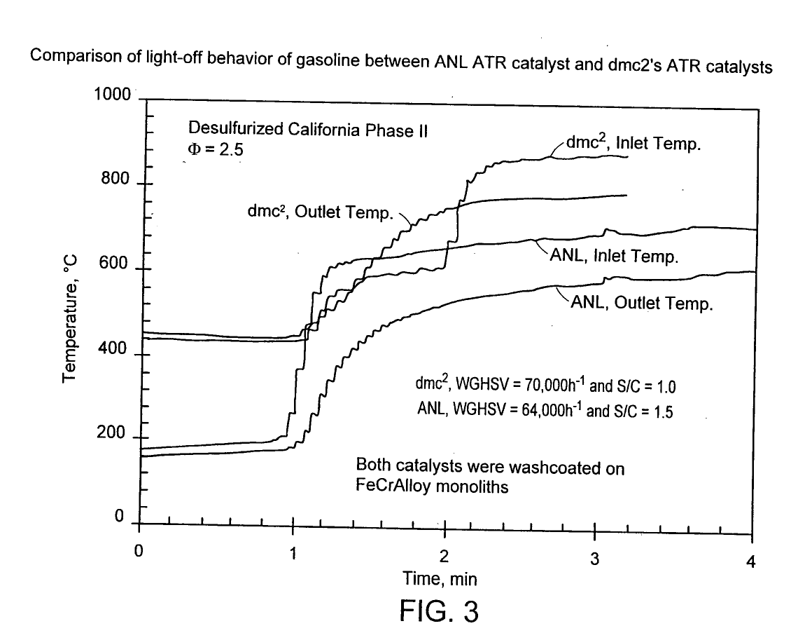

FIG. 3 compares a low-lightoff temperature catalyst with a high-lightoff temperature catalyst, both coated onto metal monoliths. Operation is at a higher space velocity than in FIG. 2, and at phi=2.5 for fast startup. The inlet and outlet temperatures of the low lightoff catalyst start to rise when the inlet temperature of the gas reaches about 200 deg. C. In contrast, the high lightoff catalyst is inactive until the inlet feed is preheated to over 400 deg. C., after which it also lights off promptly and shows rising temperatures both at the inlet and the outlet of the bed. (The humped behavior of the high temperature catalyst's inlet temperature is a feature of this particular catalyst.)

Using pairs of these or other catalysts, with a low lightoff catalyst followed by a high lightoff catalyst, it will be possible to demonstrate that a sequential use of a low-lightoff ATR catalyst followed by a high-lightoff ATR catalyst with high selectivity gives the desired effect, i.e., a catalyst bed that has both low lightoff and high selectivity, without uneconomic increase in volume. The exact identity of the catalysts giving these separate effects (respectively low lightoff temperature, and high selectivity and aromatics conversion) is not believed to be critical, and other catalysts useful for ATR and having similar properties could be substituted.

As a practical matter, the low-lightoff catalyst should have a lightoff temperature that is at least about 25 deg. C. lower than the lightoff temperature of the other catalyst, and larger lightoff temperature differentials, such as 50 deg C., 75 deg C., 100 deg C., 150 deg C., and 200 deg. C. or more, are preferred.

Likewise, there may be some increase in the total amount of catalyst required, to the extent that the low lightoff catalyst is less efficient overall. The lowered efficiency will be partially compensated by the flow of fuel enriched in the difficult species to the catalyst that reforms these species most efficiently. Thus, the overall increase in total catalyst needed in the reactor will be less than 50%, more typically less than 25%, and in favorable instances less than 10%, when compared to a catalyst consisting only of the efficient, high lightoff species.

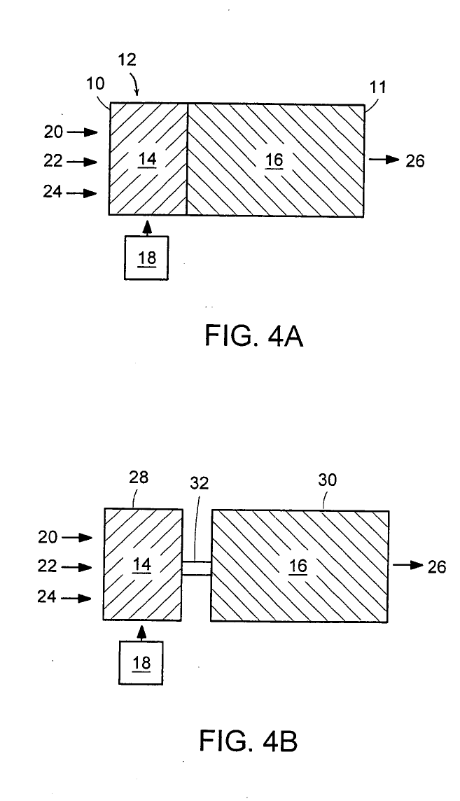

Examples of an autothermal reformer (ATR) embodying the principles of the present invention are shown in FIGS. 4A and 4B. FIG. 4A is a schematic of an autothermal reformer with an upstream catalyst portion 14 and a downstream catalyst portion 16 enclosed in a common housing. The upstream catalyst portion 14 has a first catalyst with a comparatively low light-off temperature, and the downstream portion 16 has a second catalyst with a higher light-off temperature. It will be understood that the catalysts can be mixed in the housing to some extent, and in some embodiments, the composition of the catalysts can be graded within the housing. In general, however, the low light-off catalyst forms the major component at the upstream end 10 of the housing, and the high light-off catalyst is the major component at the downstream end 11 of the housing.

During normal operation of the autothermal reformer, fuel 20, steam 22 and also a controlled amount of air 24 are optionally premixed and then injected into the upstream end 10 of the housing 12. The oxygen in the air reacts with fuel over catalyst beds 14, 16, thereby producing heat. The heat is absorbed by the endothermic reforming reaction of fuel and steam, which occurs at the same time over the catalyst beds. Once the system is at operating temperature, the amount of heat required for the reforming reaction can be generated in a controlled way by controlling the ratio of the inlet air to the amount of fuel being reformed.

During system startup, the system must initially be heated, so that the catalysts reach their effective operating temperatures. A preheat mechanism 18 is arranged to heat the catalyst(s) to their respective light-off temperature(s), after which the autothermal reaction becomes largely self-sustaining. Any conventional preheat means can be employed, such as by preheating the catalysts with hot gas, electrically heating the beds, or by heat transfer from a combustion reaction, such as from an external burner. As the reactor is initially heated by another source, such as hot gas from a burner, the low light-off catalyst in the first portion 14 will begin to operate much sooner than will the high light-off catalyst. The heat created by the oxidation reaction over the first catalyst is sufficient to maintain or increase the catalyst's activity, as well as to quickly-heat the second downstream catalyst to its light-off temperature. The reformer thus reaches normal operating conditions significantly faster than in conventional ATR reactors.

FIG. 4B shows a similar reactor, except in this case, the reactor comprises two separate housings, 28 and 30, connected by a flow path 32 for the passage of gas from the first housing 28 to the second housing 30. The first, lower light-off temperature catalyst 14 forms the major component of the catalyst bed in the upstream housing 28, and the second, higher light-off temperature catalyst 16 forms the major component of the catalyst bed in the downstream housing 30. The operation of this embodiment is substantially the same as described in connection with FIG. 4A.

While this invention has been particularly shown and described with references to preferred embodiments thereof, it will be understood by those skilled in the art that various changes in form and details may be made therein without departing from the scope of the invention encompassed by the appended claims.

1. A catalyst system for rapid startup of an autothermal reformer, the catalyst system comprising a first, upstream portion having at least a majority of a first catalyst having a first lightoff temperature, and a second, downstream portion having at least a majority of a second catalyst having a second, higher lightoff temperature, wherein the difference between the first and second lightoff temperatures is at least about 25 deg. C.

2. The catalyst system of claim 1 wherein the first and second catalyst portions are provided in a first housing and a second housing, the first housing being upstream of the second housing, the housings connected by a flow path for the passage of gas from the first housing to the second housing.

3. The catalyst system of claim 1 wherein the first and second catalyst portions are provided in a common housing, with the first catalyst being upstream of the second catalyst.

4. The catalyst system of claim 1 wherein each of the first and second catalyst portions comprises a mixture of a low-lightoff temperature catalyst and a high-lightoff temperature catalyst, the first catalyst portion comprising a higher percentage of low-lightoff temperature catalyst than the second catalyst portion.

5. The catalyst system of claim 1 wherein the difference between the first and second lightoff temperatures is at least about 50 deg. C.

6. The catalyst system of claim 5 wherein the difference between the first and second lightoff temperatures is at least about 75 deg. C.

7. The catalyst system of claim 6 wherein the difference between the first and second lightoff temperatures is at least about 100 deg. C.

8. The catalyst system of claim 7 wherein the difference between the first and second lightoff temperatures is at least about 200 deg. C.

9. A method for providing rapid startup in an autothermal reforming reaction comprising:
providing a first catalyst portion for conducting an autothermal reforming reaction, the first catalyst portion having a first lightoff temperature;
providing a second catalyst portion for conducting an autothermal reforming reaction in fluid communication with the first catalyst portion, the second catalyst portion having a second lightoff temperature that is at least 25 deg. C. higher than the first lightoff temperature;
heating at least part of the first catalyst portion to the first lightoff temperature; and
flowing a mixture comprising at least air and fuel over the heated first catalyst portion to create heat by reaction of the air and fuel.

10. The method of claim 9, further comprising adding steam to the mixture of air and fuel to allow reforming of fuel over at least the first catalyst portion to produce hydrogen.

11. The method of claim 10, wherein the steam is added before the first catalyst portion has reached the first lightoff temperature.

12. The method of claim 10, wherein the steam is added after the first catalyst portion has reached the first lightoff temperature.
